(19) United States
(12) Patent Application Publication
(10) Pub. No.: US 2003/0208250 A1
(43) Pub. Date: Nov. 6, 2003

(54) Treatment of eustachian tube dysfunction by application of radiofrequency energy

(76) Inventors: Stuart Denzil Edwards, Corral De Tierra, CA (US); Robert B. McLaughlin Jr, Sacramento, CA (US)

Correspondence Address: GLENN PATENT GROUP, 3475 EDISON WAY, SUITE L, MENLO PARK, CA, 94025, US

(21) Appl. No.: 10/168,118

(22) PCT Filed: Dec. 15, 2000
(86) PCT No.: PCT/US00/34092

(30) Foreign Application Priority Data
Dec. 15, 1999 (US) 60171021

(51) Int. Cl. A61F 7/00; A61F 7/12

(52) U.S. Cl. 607/99

(57) ABSTRACT

An atrioventricular valve tissue ablation catheter (2), especially suited for treating Mahaim fibers, includes a shaft (4) with a deflectable tip (20) at the distal end (6) and a handle (10) at the proximal end (8). The tip includes a distal segment (66) curving in one direction and a proximal segment (44) curving in the opposite direction so the distal segment causes the distal segment can engage tissue on either side of the annulus (92) of the tricuspid (or mitral) valve (90). Ablation energy can be supplied through the ablation electrodes (46, 70) simultaneously or one at a time to ablate tissue at the annulus without the need for moving the catheter. Mapping electrodes (38) are provided proximal of the ablation electrodes.

### RELATED APPLICATION

This application is a continuation-in-part of U.S. Provisional Patent Application Serial No. 60/171,021, filed Dec. 15, 1999, and entitled “Treatment of Eustachian Tube Dysfunction by Application of Radiofrequency Energy,” which is incorporated in its entirety herein by reference.

### FIELD OF THE INVENTION

This invention relates to treatment of Eustachian tube dysfunction.

### BACKGROUND OF THE INVENTION

Diseases of the middle ear, such as otitis media, mastoiditis, cholesteatoma, tympanic membrane perforation, atelectasis and other related disorders are common manifestations of Eustachian tube dysfunction. In many instances, the ventilatory function of the Eustachian tube is impaired because the tube is collapsed or occluded. In other cases, the drainage and clearance functions of the Eustachian tube are impaired because the tube is hypercompliant.

Treatment for many of these diseases is commonly at directed problems caused by hypercompliance or collapse of the Eustachian tube, rather than the dysfunction of the tube. These treatments include pharmaceutical treatments (used both curatively and as a chemoprophylactic measure) and surgical treatments such as myringotomy, adenoidectomy and placement of tubes.

These remedies are imperfect at best. The benefits derived from surgical treatment are transient: on average, problems recur after twelve months. This recurrence leads to further treatment and increased risk of complications.

Eustachian tube dysfunction is the most important factor in the pathogenesis of middle ear disease. Otitis Media is the second most common disease of childhood. Two thirds of children have at least one episode of otitis media by age three years. After an episode of acute otitis media, 10% of children have middle ear effusion lasting three months or more. See FIGS. 1,2. Prolonged middle ear effusion associated with hearing loss may interfere with speech and language development. The annual reports of Vital and Health statistics published for 1993 by the National Center for Health Statistics of the Centers for Disease Control and Prevention estimated 27.845 million cases of acute ear infection in the United States. 14.751 million occurred in children younger that 5 years of age, and 7.501 million occurred in children between the ages of 5-17 years of age. The remaining 6 million occurred in adults 18 years of age and older. The AHCPR panel estimates that 25-35% of the total cases represented otitis media with effusion.

The management of otitis media is initially medical, with surgical therapy reserved for medical failures or complications. Failure of medical and surgical therapy has been associated with persistent Eustachian tube dysfunction. Complications include hearing loss, speech delay, tympanic membrane perforations, atelectasis, retraction pockets, cholesteatoma, mastoiditis as well as less common life threatening intracranial extensions of infection.

The current surgical standard of care, myringotomy and tubes, fails to address the underlying pathophysiology of Eustachian tube dysfunction, resulting in temporary benefit (average 12 months) and the need for repeat procedures (40%). The following document outlines the clinically relevant anatomy and physiology of Eustachian tube dysfunction and explores the potential market opportunity for treatment of this disorder.

A unique technological device is proposed. In light of the large target population, in addition to growth in general otolaryngology, the proposed device would serve as a catalyst for future growth into Otology and Pediatric Otolaryngology. Additional devices could then be developed based to complement the otology and pediatric otolaryngology suites.

Accordingly, it would be advantageous to provide an improved technique for treating Eustachian tube dysfunction. This advantage is achieved by a method and system whereby the Eustachian tube dysfunction is treated by manipulating the adjacent structures so as to strengthen, support and decrease tissue bulk in the Eustachian tube vault.

### SUMMARY OF THE INVENTION

The invention provides a method and system for treatment Eustachian tube.

In a first aspect of the invention, the device includes a component that identifies and targets specific tissues proximate to the Eustachian tube. Tissues that can be manipulated in a manner so as to enhance the structural integrity of the Eustachian tube are targeted. This targeting may involve stimulation of muscles or nerves. These tissues may include the tensor veli palatini, the salpinopharyngeal and the levator veli palatini. In some patients, the tensor tympani may also be targeted.

In a second aspect of the invention, the device includes a feedback element that measures the pressure differential across the middle ear. This feedback is used to determine the end point of treatment. In a preferred embodiment, feedback will be obtained with an intact tympanic membrane. However, in other embodiments, myringotomy may be necessary to achieve sufficient feedback. Since this treatment is relative noninvasively and does not require sedation, additional feedback may be obtained by asking the patient how they feel at various points during a treatment.

In a third aspect of the invention, very low levels of RF energy are applied using either a bipolar or monopolar electrode to muscles that determine the structural integrity of the Eustachian tube. Targeted areas are heated to a temperature between 60 and 70 degrees Celsius. Application of this energy shrinks collagenaeous, fibers and shortens tendons, This indirectly decreases the compliance of the midportion of the cartilaginous Eustachian tube and biases it in favor of opening.

In a preferred embodiment, the RF electrodes are inserted orally and treatment is directed through the palate. However, in alternate embodiments, the electrodes may be inserted transnasally, using endoscopic visualization.

Features and advantages of the inventions are set forth in the following Description and Drawings, as well as in the appended Claims.

### BRIEF DESCRIPTION OF THE DRAWINGS

FIGS. 1 to 6 illustrate the problems and solutions that the invention addresses.

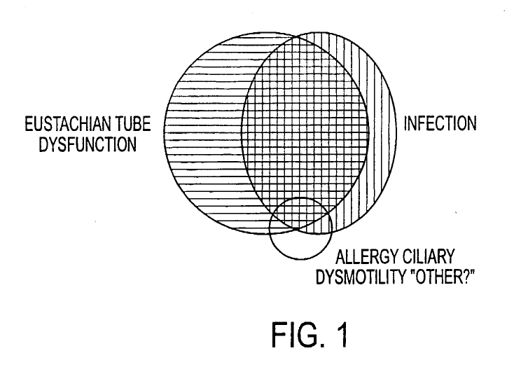

The invention may be embodied in several forms without departing from its spirit or essential characteristics. The scope of the invention is defined in the appended claims, rather than in the specific description preceding them. All embodiments that fall within the meaning and range of equivalency of the claims are therefore intended to be embraced by the claims.

### DETAILED DESCRIPTION OF THE PREFERRED EMBODIMENTS

In the following description of the invention is described with regard to preferred process steps and structures. Those skilled in the art would recognize, after perusal of this application, that embodiments of the invention can be implemented using circuitry or other structures adapted to particular process steps and structures, and that implementation of the process steps and structures described herein would not require undue experimentation or further invention.

I. Anatomy of the Eustachian Tube:

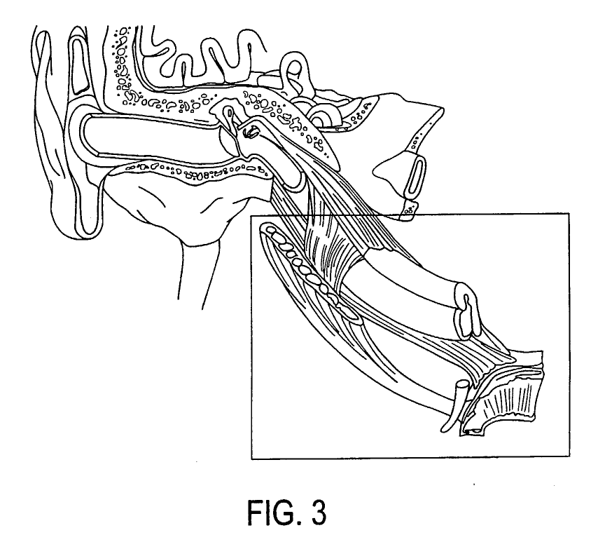

The Tube: The lumen is wider at both the proximal(nasopharyngeal) and distal (middle ear) ends than the mid portion (isthmus) where it is narrowest. The Eustachian tube (ET) is longer in the adult (31-38 mm) than in the infant and young child. By 6 years of age, the ET has generally attained adult proportions. Tubal cartilage increases in mass from birth to puberty and this development has physiologic implications. In the adult, the posterior third is osseous; and the anterior two-thirds is cartilaginous. The healthy osseous portion is open at all times, in contrast to the fibrocartilagenous portion which is closed at rest, but opens during swallowing. See FIG. 3. The cartilaginous tube courses anteromedially and inferiorly to insert into the nasopharynx at the superior border of the superior constrictor where it forms the torus tubarious. The mucosal lining is respiratory epithelium (pseudocolumnar ciliated) that is continuous with the middle ear and nasopharynx.

Peritubal musculature: Four muscles are generally cited as being associated with the Eustachian tube.

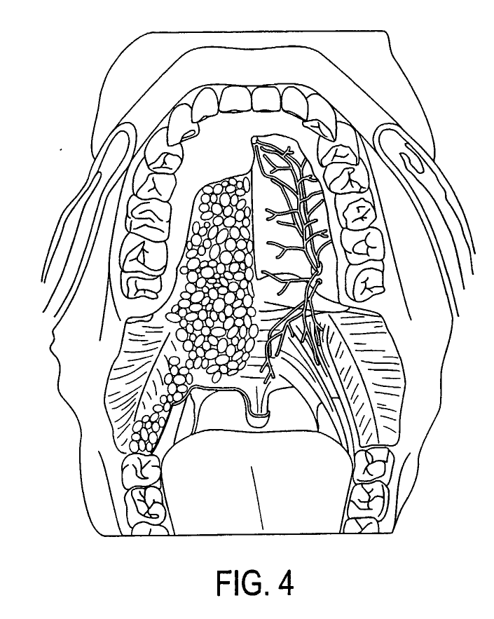

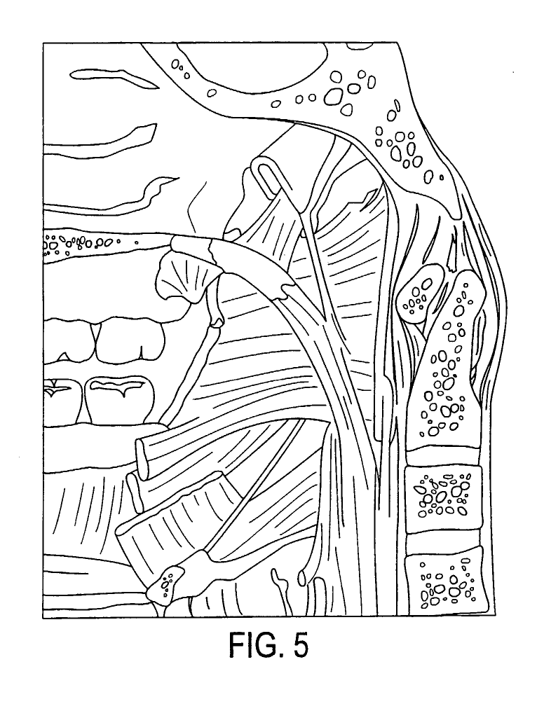

The tensor veli palatini is composed of two distinct bundles of muscle fibers. The tensor veli palatini proper is an inverted triangle with its bony insertions (fixed) in the scaphoid fossa and entire lateral osseous ridge of the sulcus tubae for the course of the eustachian tube. The medial bundle (dilator tubae muscle) inserts into the posterior third of the lateral membranous wall of the Eustachian tube. It is responsible for active dilation of the tube by inferolateral displacement of the membranous wall. Both bundles descend to converge in a tendon that passes under the hamulus of the pterygoid bone to its origin in the posterior border of the horizontal process of the palatine bone (hard palate) and the palatine aponeurosis of the anterior portion of the velum. See FIGS. 3, 4, 5.

The tensor tympani (not veli) is a separate muscle slip originating from the tubal cartilages and sphenoid bone and inserts into the manubrium of the malleus. It does not appear to be involved in Eustachian tube function.

The salpingopharyngeal muscle arises from the medial and inferior borders of the tubal cartilage. It is composed of thin slips of muscular and tendinous fibers. Generally thought to lack the ability to perform physiologically.

The levator veli palatini originates from the inferior petrous apex and the lower border of the medial lamina of the tubal cartilage. It runs along the floor of the eustachian tube to insert in the dorsal surface of the palate. The muscle is not an active dilator of the tube, rather adds support via its relationship to the tube by loose connective tissue.

II. Physiology of the Eustachian Tube

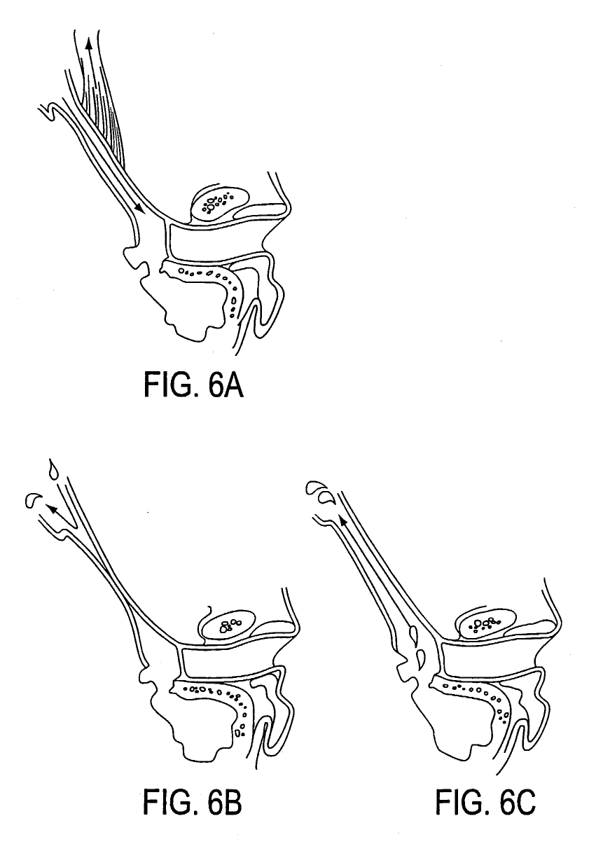

The Eustachian tube has three primary functions with respect to the middle ear: (1) ventilation of the middle ear space with respect to atmospheric pressure, (2) protection from nasopharyngeal sound, pressure and secretions, and (3) drainage and clearance of middle ear secretions into the nasopharynx. See FIG. 6.

1. The Ventilatory Function is the most important function of the Eustachian tube without which the other functions are impaired as well. Ideal tubal function is thought to be intermittent active opening of the eustachian tube via contraction of the tensor veli palatini during swallowing in order to maintain nearly ambient pressures in the middle ear. When active tubal function is ineffective, functional collapse of the tube persists which results in negative pressure in the middle ear.

2. Protective, drainage and clearance functions: The compliance of the cartilaginous midportion of the eustachian tube seems to be the most critical factor with regard to these functions. For example: Sudden drops in middle ear pressure with an overly compliant tube can lock the Eustachian tube closed. In this case, a perforation or myringotomy would release the lock and promote return of function. However, if the compliance and tensor veli palatini contraction was able to counteract the negative pressure the problem may not have developed in the first place. Inpatients with recurrent or chronic middle ear effusions despite treatment with myringotomy and tubes the problem may result from either a hypercompliant ET with or without (cleft palate) an effective tensor veli palatini to overcome the negative middle ear pressure. Other factors such as mitcociliary function, the length of the midportion, active pump via tubal opening and closing, and surface tension factors are critical considerations as well. If the tube is hypercompliant and prone to collapse with negative middle ear pressure then all of these factors would be impaired. Inflammation and mechanical impingement would cease mucociliary flow. The pump would be impaired by the inability to overcome the negative pressure lock, and surface tension factors would become more significant based on the higher ratio of surface area to volume in a narrowed tube. This is consistent with observations by Honjo in which he demonstrated the Eustachian tube to pump liquid out of the middle ear in humans however when negative pressure was applied to the middle ear the function was impaired.

Eustachian tube dysfunction appears to be the most important factor in the pathogenesis of middle ear disease. Other contributing factors include infection of the respiratory mucosa, allergy, ciliary dysfunction.

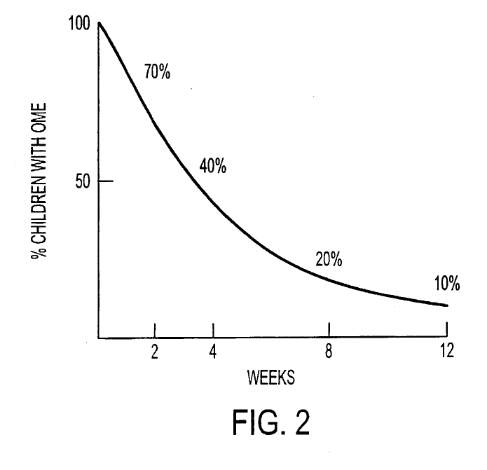

Acute Otitis Media (AOM): In studies by Teele and Co-workers, by age 12 months, 60% of all children had at least one episode of AOM. By age 3 years, nearly 50% had at three or more episodes of acute otitis media. Otitis prone children seem to have their first episode within the first year of life. Uncomplicated cases treated with oral antibiotics. Up to 50% of children will have a persistent middle ear effusion (MEE). Treatment options for the acute MEE including additional antibiotics, decongestants, steroids, and Eustachian tube inflation have not been shown to be significantly more effective than observation in randomized studies. FIG. 2 shows the duration of effusion after a single episode of acute otitis media. The mean duration was 40 days in one study. 10% persist to have MEE as long as 3 months (chronic condition) after resolution of the acute process.

Recurrent Acute Otitis Media (RAOM): In the absence of persistent middle ear. effusion recurrent bouts of acute otitis media should be considered a sign of other disease processes. Treatment options include chemoprophylaxis, myringotomy and tube insertion. adenoidectomy was recently shown in a randomized trial to not significantly impact the incidence of recurrent acute otitis media.

Otitis Media with Effusion (OME): Investigations of healthy children have revealed a high incidence of asymptomatic middle ear effusion with a peak incidence in the 2nd year of life. In a study of children aged 2-5 years, 53% of children during the first year of the study and 61% during the second year developed OME. Teele found that the mean time with OME was on average I month for each year of the first two years of life. Some children had middle ear fluid more than half the length of their infancy, however most resolve without medical or surgical intervention. 10% of all bouts of AOM are followed by MEE lasting greater than three months. Treatment options for the acute MEE including additional antibiotics, decongestants, steroids, and Eustachian tube inflation have not been shown to be significantly more effective than observation in randomized studies. Surgical options are usually reserved for. patients with symptoms that do not resolve with observation or medical therapy. Myringotomy and Tubes with and without adenoidectomy has been shown to improve conductive hearing loss secondary to OME and to decrease the amount of time spent with a MEE.

Tympanic Membrane Atelectasis: Focal retraction pockets can result in entrapment of desquamation byproducts resulting in cholesteatoma formation and ossicular erosion. Diffuse tympanic membrane flaccidity is often referred to as an atelectatic tympanic membrane and occurs in 2% of patients with persistent otitis media. The underlying pathophysiology results from Eustachian tube dysfunction causing on of the tympanic membrane results in remodeling of the tympanic membrane resulting in a persistent state of flaccidity despite treatment of the negative middle ear pressures. A second procedure to treat the persistent atelectatic tympanic membrane will be discussed later in proposed procedures.

Chronic Otitis Media: Tympanic membrane perforation after tube placement (3% incidence after ear tubes) and recurrent or persistent perforations after tympanoplasty (5% failure rate) appear to be related to chronic Eustachian tube dysfunction. Acquired cholesteatomas, recurrent retraction pockets, poor mastoid aeration and atelectatic tympanic membranes have also been reported to occur in relation to Eustachian tube dysfunction.

Cleft Palate: Incidence in 1 in 750 births. Stool and Randal reported a 94% incidence of middle ear effusion at the time of myringotomy in cleft plate infants. Children with any form of unrepaired palatal cleft (including bifid uvula, submucous cleft) have a higher incidence of otitis at all ages. The incidence of middle ear disease decreases somewhat after surgical repair of a cleft palate and seems to be related to anatomic correction of the tensor veli palatini.

Craniofacial Anomalies: Down's syndrome 1 in 1000 births. Any abnormality of the midface, palate or skull base can cause Eustachian tube dysfunction including Treacher Collins syndrome, Apert's syndrome, and the mucopolysaccharidosis.

Complications of Otitis Media: Intratemporal complications include hearing loss (conductive and sensorineural), acute and chronic tympanic membrane perforations, chronic suppurative Otitis Media with and without cholesteatoma, retraction pockets and atelectasis, adhesive otitis media, tympanosclerosis, ossicular fixation and discontinuity, Mastoiditis, petrositis, labrynthitis, and facial paralysis. Intracranial complications including Meningitis, subdural empyema, brain abscess, extradural abscess, lateral sinus thrombosis and otic hydrocephalus are unusual, but have serious potential neurologic sequelae including death.

Complications of Myringotomy and tubes: Otorrhea is the most common requiring treatment with topical and systemic antibiotics. Avoidance of water exposure is necessary. The average tube lasts approximately 9-15 months. Replacement or repeat tube placement comprises 30% of yearly ear tube procedures. Approximately 10% fail to extrude and must be removed. Other complications include tympanic membrane perforations (3%) and cholesteatomas (1%).

Complications of Adenoidectomy: Also rare, however include severe complications including bleeding (1%) nasopharyngeal stenosis and Eustachian tube injury.

The prevalence of eustachian tube dysfunction is not known. There are not reliable objective measures that can be routinely used to screen normal patients for the presence of ETD. Otologic manifestations of the disorder as outlined seem. to be the best measure of its prevalence relative to clinical significance and will be expanded upon the discussion that follows. However, there is population of adults that present with chronic and recurrent complaints of aural fullness, hearing loss, imbalance, or tinnitis in an otherwise normal appearing ear. The symptoms frequently resolve with autoinsufflation (ETD). This clinical entity frequently fails to respond to medical therapy including nasal steroids, decongestants, antibiotics, allergy evaluation and immunotherapy. There are no broadly accepted surgical therapies for this disorder. The demographics of this patient population are poorly characterized in the literature. Elner et al. studied otologically normal adults ability to, equilibrate static positive and negative middle ear pressures of 100 mm H2O. They showed that 95% of normal adults could equilibrate an applied positive pressure, and that 93% could equilibrate applied negative pressure to some degree. However, 28% of adult subjects could not completely equilibrate either applied positive or negative pressure or both. Therefore, anywhere from 5-28% of the otologically normal (by physical exam) adult population may exhibit symptoms of eustachian tube dysfunction. A UK national study estimated that the prevalence of eustachian tube dysfunction in adults (18-80 years of age) is 0.9%.

Prevalence of Otitis media with effusion: Otitis media with effusion is the most common disease treated by physicians who care for children and the second most common indication for surgery in children (after circumcision).

The annual reports of Vital and Health statistics published for 1993 by the National Center for Health Statistics of the Centers for Disease Control and Prevention estimated 27.845 million cases of acute ear infection in the United States. 14.751 million occurred in children younger that 5 years of age, and 7.501 occurred in children between the ages of 5-17 years of age. The remaining 5.593 million occurred in adults 18 years of age and older. This includes both primary and recurrent cases in 1993. As reported by Teele, 10% of cases of AOME persist beyond 3 months giving us an estimate of 1.4 million cases for children less than 5 years of age in 1993. With regards to chronic otitis media with effusion, the true prevalence is not known.

Health Care Expenditures:

A conservative estimate of cost was derived by Gates. His estimate used 2.5 physician visits at 35$ and 1.5 courses of antibiotics (Amoxacillin) for a direct cost of $100 and indirect costs based on a loss of one-half day of work (43.00) plus travel expenses of 10.00 per visit. Projecting these costs on 14 million results in annual expenditures of 3.15 billion dollars for the care of AOME in children under the age of 5 years. Estimates with regard to children greater than the age of 5 years are more difficult to derive, however treatment of recurrent episodes engenders substantial health care costs for the small group of patients with multiple recurrences. (Gates)

Using similar assumptions as found in the AHCPR commission report for 1993 Gates extrapolated these modified assumptions on the basis of a COME rate of 10% for the 14 million cases of AOME in children with AOME younger than 5 years of age. Direct costs were estimated at $1.484 billion and indirect costs of 0.370 billion for a total cost of 1.854 billion. His total estimate with respect to a total cost of 5 billion dollars spent on indirect and direct costs of treating OME only included children under 5 years of age. (Table 1)

In the Unites States >25% of the estimated 120 million prescriptions written for oral antibiotics each year are for the treatment of otitis media. A more recent survey of antimicrobial drug prescribing among office based physicians revealed that not only is the incidence of prescribing for otitis media increasing, there has also been an increase in the use of broader spectrum and more expensive antibiotics which may be contributing to a bacterial resistance problem.

In 1988, using federal data, approximately 800,00 children received 1.3 million tympanostomy tubes. Of these 30% were replacements. In 1994, 45,000 sets of ear tubes were placed in conjunction with adenoidectomy, and 140,000 US children less than 15 years of age underwent adenoidectomy with most having a primary or secondary diagnosis of otitis; media.

In 1991, the AHCPR Commission estimated that 821,700 cases of Otitis media with effusion occurred in 2 y/o children in 1991, and that 1.09 billion dollars was spent for direct and indirect costs of care in these cases. They found that 52 percent of 2 y/o children were managed without surgery with an average of 4.6 office visits and 25% incurring hearing tests for an average cost of $406.00 per. patient. 42% of children in the sample studied underwent myringotomy and tubes insertion with an average of 5.5 office visits and 40% incurring hearing tests for an average cost of $21 74. 00 per patient. 6% of these children underwent adenoidectomy with an average of seven office visits for pre and postoperative visits, 45 percent underwent hearing tests and $3433. 00 was spent per patient.

Ultimately, the proposed procedure would target patients that suffer from eustachian tube dysfunction unresponsive to medical therapy. This would include adult and pediatric patient populations. Because we do not yet know the effects of radiofrequency treatment on eustachian tube and peritubal muscular development, the pediatric patient should be considered as being composed of two primary groups based on a cutoff age at which an adult-like eustachian tube is developed. Lets say 5 years of age for ease of interpreting above data, although 6 or 7 years of age is probably more accurate. In all three patient populations (two pediatric groups and adults), chronic otitis media with effusion and recurrent otitis media would be relative indications for the proposed procedure. Cleft and Craniofacial pediatric patients known to be at very high risk of chronic eustachian tube dysfunction should be considered separately. The anticipated pediatric (<6 years of age) patient population would include the otitis prone child, children with recurrent otitis media and chronic otitis media with effusion refractory to medical therapy. In the case of otitis media with effusion, patients may be considered for intervention either primarily or after recurrence with more than one set of myringotomy tubes. In children younger than 5 years of age in 1993, 14 millions incidences of acute otitis media with effusion occurred. Ten percent or 1.4 million cases persisted beyond three months at which time surgical intervention should be considered based upon symptoms of hearing loss and bilaterality of disease. If the proposed procedure was utilized only after treatment failure with at least one set of ear tubes, based upon 30% of 1.3 million myringotomy and tubes procedures were for replacement tubes then we derive a target population of 390,000 each year for this age group. (Table 1)

In 1993, 7.501 million cases of otitis media occurred in children between the ages of 6-17 years of age. The incidence of chronic otitis media with effusion and recurrent otitis media that occurs in this population is poorly characterized. Most cases of acute otitis media resolve and acute otitis media with effusion resolve spontaneously in this patient population. However, within this patient population, there is a clear subset of patients that persist to suffer chronic ear disease requiring multiple sets of myringotomy tubes and chronic ear surgery. If one assumes a 3% rate of persistent tympanic membrane perforations and a I% incidence of cholesteatomas after myringotomy and tube placement (Golz), then 1.3 million myringotomy and tubes performed each year is associated with 39,000 persistent perforations and 13,000 cholesteatomas each year. In addition, the most common cause of failed tympanoplasty (5%) and recurrent cholesteatoma is eustachian tube dysfunction. If one extrapolates from the prevalence of active chronic otitis media (1.5%) and the incidence of eustachian tube dysfunction(0.9%) demonstrated in the UK study, then based on the 1996 US census estimates for this age group 858,435 and 515,061 patients suffer from active chronic otitis media and eustachian tube dysfunction respectively. Table 2.

The remaining 5.593 million incidences of otitis media in 1993 occurred in adults 18 years of age and older. Again, the incidence of chronic otitis media with effusion and recurrent otitis media that occurs in this population is poorly characterized. If one extrapolates from the prevalence of active chronic otitis media (1.5%) and the incidence of eustachian tube dysfunction (0.9%) demonstrated in the UK study, then based on the 1996 US census estimates for this age group 2.9 million and 1.7 million patients suffer. from active chronic otitis media and eustachian tube dysfunction respectively. (Table 3)

Children with cleft palate or other craniofacial abnormalities that effect eustachian tube dysfunction occur in an estimated 1:750 births utilizing estimates based solely on the incidence of cleft palate (most common) to avoid overlap. Based on the 4.1 million births in the United Stated in 1990 we can derive an approximate target population of 5400 newborns each year with craniofacial defects strongly associated with eustachian tube dysfunction each year. Furthermore, it is estimated based on the 1996 US census population estimates that there is a cleft patient pool of 353,000. (Table 4)

Patients with persistent Eustachian tube dysfunction utilize significant resources with regard to pharmacotherapy, numerous sets of ear tubes, adenoidectomy, in addition complications related to chronic otitis media as well as the indirect costs of a active chronic otitis media (1.5%) and the incidence of eustachian tube dysfunction (0.9%) demonstrated in the UK study, then based on the 1996 US census estimates for this age group 858,435 and 515,061 patients suffer from active chronic otitis media and eustachian tube dysfunction respectively. Table 2.

The remaining 5.593 million incidences of otitis media in 1993 occurred in adults 18 years of age and older. Again, the incidence of chronic otitis media with effusion and recurrent otitis media that occurs in this population is poorly characterized. If one extrapolates from the prevalence of active chronic otitis media (1.5%) and the incidence of eustachian tube dysfunction (0.9%) demonstrated in the UK study, then based on the 1996 US census estimates for this age group 2.9 million and 1.7 million patients suffer. from active chronic otitis media and eustachian tube dysfunction respectively. (Table 3)

Children with cleft palate or other craniofacial abnormalities that effect eustachian tube dysfunction occur in an estimated 1:750 births utilizing estimates based solely on the incidence of cleft palate (most common) to avoid overlap. Based on the 4.1 million births in the United Stated in 1990 we can derive an approximate target population of 5400 newborns each year with craniofacial defects strongly associated with eustachian tube dysfunction each year. Furthermore, it is estimated based on the 1996 US census population estimates that there is a cleft patient pool of 353,000. (Table 4)

Patients with persistent Eustachian tube dysfunction utilize significant resources with regard to pharmacotherapy, numerous sets of ear tubes, adenoidectomy, in addition complications related to chronic otitis media as well as the indirect costs of a chronic illness. A treatment that was aimed at the underlying cause of the problem, Eustachian tube dysfunction could make significant strides toward prevention of future recurrences and significant cost savings.

Patient requirements: Minimally invasive, minimal risk, longterm results, quick recovery, and outpatient/ambulatory procedures. The proposed procedure is equal to the current surgical standard (myringotomy and tubes) in every way and should provide added benefits of improved longevity, fewer/less severe complications, and fewer repeat and adjunctive procedures.

Targeted users: General Otolaryngologists including specialty trained pediatric otolaryngologists, otologists, and neurotologists.

The invention provides a method and system for treatment of Eustachian tube dysfunction. In a first aspect of the invention, the device includes a component that identifies and targets specific tissues proximate to the Eustachian tube. Tissues that can be manipulated in a manner so as to enhance the structural integrity of the Eustachian tube are targeted. This targeting may include the tensor veli palatini, the salpinopharyngeal and the levator veli palatini. In some patients, the tensor tympani may also be targeted. In a second aspect of the invention, the device includes a feedback element that measures the pressure differential across the middle ear. This feedback is used to determine the end point of treatment. In a preferred embodiment, feedback will be obtained with an intact tympanic membrane. However, in other embodiments, myringotomy may be necessary to achieve sufficient feedback. Since this requirement is relative non-invasive and does not require sedation, additional feedback may be obtained by asking the patient how they feel at various points during a treatment. In a third aspect of the invention, very low levels of RG energy are applied using either a bipolar or monopolar electrodes to muscles that determine structural integrity of the Eustachian tube. Targeted areas are heated to a temperature between 60 and 70 degrees Celsius. Application of this energy shrinks collagenous fibers and shortens tendons. This indirectly decreases the compliance of the midportion of the cartilaginous Eustachian tube and biases it in favor of opening. In a preferred embodiment, the RF electrodes are inserted orally and treatment if directed through the palate. However, in alternate embodiments, the electrodes may be inserted transnasally, using endoscopic visualization.

Proposed Procedure:

Type I Collagen is the primary component of tendonous and ligamentous structures. The application of heat to collagen results in 25%-20% contraction and remodeling of collagen. The reaction is temperature dependent occurring maximally between 60-70 degrees Celsius. The reaction can be achieved at a slower rate and in a more precise fashion by treating at a lower temperature for a sustained, precise treatment time. The proposed procedure uses very low levels of radiofrequency energy to shrink the collagen or shorten the tendon of the tensor veli palatini (palate) from its origin in the palate toward the hamulus. These lesions will be resorbed and remodeled resulting in a shortened tendon and improved muscle tone and force of contraction. By effectively shortening the tensor veli palatini, we are indirectly decreasing the compliance of the midportion of the cartilaginous Eustachian tube and improving the opening force of contraction. Treatment at the palatal origin is the most accessible, however a transnasal approach under endoscopic visualization would be possible as well.

The envisioned device will have three primary components:

1. Bipolar versus monopolar RF applicator.

2. Targeting system: Muscle stimulator

3. Feedback system: Concurrent transtympanic Eustachian tube testing.

Other possible treatments might include treating the salpingopharygeous; or the fascia and constrictors of the nasopharynx, i.e. creating functional medial scar contracture. Treating the levator veli palatini to effectively strengthen support of or ablation to decrease tissue bulk in the confines of the Eustachian tube vault. Treating the Eustachian tube or the immediate surrounding structures to decrease compliance. However treatment in these cases in the region of the ET vault could prove hazardous due to the adjacent carotid artery. In adult, all of these procedures could be performed on an outpatient basis or in a minor procedure room under local anesthesia. It is unlikely that sedation would be required. In children, general anesthesia by mask ventilation or endotracheal tube would likely be required.

A programmable radiofrequency generator with temperature and impedance monitoring and a suite of proprietary disposable electrode devices that deliver radiofrequency energy to selected areas. An insulating sleeve at the base of the needle electrode protects surrounding tissue from thermal damage. Thermocouples in the insulation and at the tip of the needle electrode assist in accurate monitoring of tissue temperature ensuring optimal ablation without excessive heat damage.

Clinical Advantages:

The primary advantage over current therapies is that the proposed procedure treats the physiologic origin, Eustachian tube dysfunction, thereby providing significantly improved longterm results. Other advantages include a significant cost saving and reduced morbidity and complication rate. It benefits from a decreased rate of repeat procedures and complications related to otorrhea, tympanic membrane perforations, cholesteatoma, and complications of otitis media. In addition it is a comparable with regard to postoperative discomfort.

Other Proposed Otologic Applications

In light of the large target population, in addition to growth in general otolaryngology, the proposed device would serve as a catalyst for future growth into Otology and Pediatric Otolaryngology. Additional devices could then be developed based on this market advantage to complement the otology and pediatric otolaryngology suites.

The atelactic tympanic membrane is caused by connective tissue remodeling occurring as a result exposure to a persistent negative middle ear pressure. Once the middle ear pressure has been treated by addressing the underlying Eustachian tube or by myringotomy and tube, a proportion of tympanic membranes will remain flaccid. Radiofrequency induced thermal contraction of the tympanic membrane should restore the normal architecture and compliance of the tympanic membrane. Thermal induced myringotomies, currently lasers, provide intermediate term myringotomies that may prove to be efficacious for some clinical indications.

Various features of the invention are set forth in the following Claims.

1. A system for applying energy to one or more tissue structures proximate to the middle ear, comprising:
means for locating and targeting a tissue structure to be treated;
means for delivering energy to said targeted tissue; and
means for monitoring treatment progress so that a treatment endpoint is determined and thermal damage to surrounding tissues minimized.

2. The system of claim 1, wherein said tissue structures include one or more of:
tensor veli palatini muscle;
levator veli palatini muscle;
salpingopharyngeal;
nasopharyngeal fascia;
nasopharyngeal constrictors;
eutachian tube vault and/or tissue structures contained therein;
tympanic membrane;
tensor tympani; and
tissues that can be manipulated so as to enhance eustachian tube structural integrity.

3. The system of claim 1, wherein said means for locating and targeting tissue structures comprises either a nerve or muscle stimulator, wherein stimulating nerves/and or muscles allows them to be visualized or located.

4. The system of claim 1, wherein said means for delivering energy to said targeted tissue comprises an RF applicator, wherein low level RF energy is applied to said tissue structures to shrink collagenous portions of said tissue structures.

5. The system of claim 4, wherein said RF applicator comprises:
one or more monopolar or bipolar electrodes.

6. The system of claim 5, wherein said one or more electrodes comprises a needle electrode having a base and a tip, said base surrounded with an insulating sleeve to protect surrounding areas from thermal damage.

7. The system of claim 6, said needle electrode including one or more thermocouples disposed in one or both of said insulation and said electrode tip.

8. The system of claim 6, wherein said needle electrode is inserted orally.

9. The system of claim 6, wherein said needle electrode is inserted transnasally, using endoscopic visualization

10. The system of claim 5, wherein said RF applicator further comprises:
a programmable RF generator, said RF generator equipped with means for monitoring one or both of temperature and impedance at a treatment site.

11. The system of claim 1, wherein said means for monitoring treatment progress comprises an element for measuring pressure differential across the middle ear.

12. A method of applying energy to one or more tissue structures proximate the middle ear, comprising the steps of:
locating and targeting the tissue structures to be treated;
delivering energy to said targeted tissue structures; and
monitoring treatment progress so that a treatment endpoint is determined and thermal damage to surrounding tissues minimized.

13. The method of claim 12, wherein said tissue structures include one or more of:
tensor veli palatini muscle;
levator veli palatini muscle;
salpingopharyngeal;
nasopharyngeal fascia;
nasopharyngeal constrictors;
eutachian tube vault and/or tissue structures contained therein; and
tissues that can be manipulated so as to enhance eustachian tube structural integrity.

14. The method of claim 12, wherein the step of locating and targeting tissue structures comprises:
stimulating one or both of nerves and muscles proximate to the middle ear, wherein stimulating said nerves/and or muscles allows them to be visualized or located.

15. The method of claim 12, wherein said step of delivering energy to said targeted tissue strucutres comprises applying low level RF energy to said tissue structures b y means of an RF applicator to shrink collagenous portions of said tissue structures.

16. The method of claim 15, wherein said RF applicator comprises:
one or more monopolar or bipolar electrodes.

17. The method of claim 16, wherein said one or more electrodes comprises one or more needle electrodes, each electrode having a base and a tip, said base surrounded with an insulating sleeve to protect surrounding areas from thermal damage.

18. The method of claim 17, each needle electrode including one or more thermocouples disposed in one or both of said insulating sleeve and said electrode tip.

19. The method of claim 17, further comprising the step of inserting said needle electrode orally.

20. The method of claim 17, further comprising the step of inserting said needle electrode transnasally using endoscopic visualization.

21. The method of claim 16, wherein said RF applicator further comprises:
a programmable RF generator, said RF generator equipped with means for monitoring one or both of temperature and impedance at a treatment site.

22. The method of claim 12, wherein said means for monitoring treatment progress comprises an element for measuring pressure differential across the middle ear.
